(19) United States
(12) Patent Application Publication
(10) Pub. No.: US 2005/0131021 A1
(43) Pub. Date: Jun. 16, 2005

(54) Phenoxy piperidines for treating diseases such as schizophrenia and depression

(75) Inventors: Gunter Holzemann, Seeheim-Jugenheim (DE); Helmut Prucher, Heppenheim (DE); Kai Schiemann, Seeheim-Jugenheim (DE); Joachim Leibrock, Pfungstadt (DE); Hartmut Greiner, Weiterstadt (DE); Christa Burger, Darmstadt (DE); laurie von Melchner, Kronberg (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstadt (DE)

(21) Appl. No.: 10/501,763

(22) PCT Filed: Dec. 17, 2002
(86) PCT No.: PCT/EP02/14389

(30) Foreign Application Priority Data
Jan. 17, 2002 (DE) 102 01 550.3

(51) Int. Cl. C07D 211/40; A61K 31/445

(52) U.S. Cl. 514/317; 514/319; 546/205; 546/216

(57) ABSTRACT

Compounds of the formula (1), in which R¹, R²′, R²″, R²′″, R³ and n are as defined in claim 1, are effectors of the nicotinic and/or muscarinic acetylcholine receprot and are suitable for the prophylaxis or treatment of schizophrenia, depression, anxiety states, dementia, Alzheimer's disease, Lewy bodies dementia, neurodegenerative diseases, Parkinson's disease, Huntington's disease, Tourette's syndrome, learning and memory impairments, age-related memory impairment, amelioration of withdrawal symptoms in nicotine dependence, strokes or brain damage by toxic compounds.

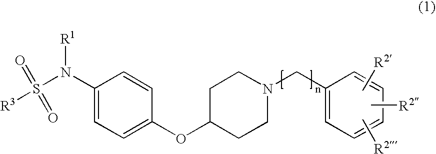

The invention relates to phenoxy-piperidines of the formula I 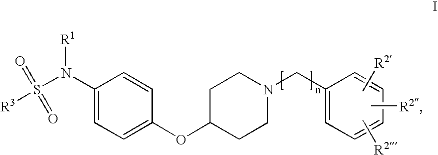
in which
R is H or A,
R²′, R²″, R²′″ are each, independently of one another, H, A, OH, OCH₃, OCF₃, Hal, CN, COOR¹, CONR¹ or NO₂,
R³ is A, Ar or A-Ar,
R⁴ is H or A,
A is unbranched or branched alkyl having 1-10 carbon atoms, in which one or two CH₂ groups may be replaced by O or S atoms and/or by —CH═CH— groups and/or 1-7H atoms may also be replaced by F,
Ar is phenyl, naphthyl or biphenyl, each of which is unsubstituted or mono-, di- or trisubstituted by Hal, A, OR⁴, N(R⁴)₂, NO₂, CN, COOR⁴, CON(R⁴)₂, NR⁴COA, NR⁴CON(R⁴)₂, NR⁴SO₂A, COR⁴,

SO₂N(R⁴)₂ or SO₂A,
A-Ar is arylalkyl, where A and Ar have one of the above-mentioned meanings,
Hal is F, Cl, Br or 1, and n is 0, 1, 2, 3, 4, 5, 6, 7, 8, 9 or 10,
   and pharmaceutically usable derivatives, solvates and stereoisomers thereof, including mixtures thereof in all ratios.

The invention had the object of finding novel compounds having valuable properties, in particular those which can be used for the preparation of medicaments.

It has been found that the compounds of the formula I and pharmaceutically usable derivatives, solvates and stereoisomers thereof, while being well tolerated, have valuable pharmacological properties since they have actions on the central nervous system. The compounds are, in particular, effectors of the nicotinic and/or muscarinic acetylcholine receptor, where they exhibit agonistic or antagonistic action.

Of the well-characterised class of acetylcholine receptors, some members have been implicated in certain disorders of the central nervous system. Known active ingredients which are able to interact with the class of acetylcholine receptors are, for example, pilocarpine, nicotine, lobeline and epibatidine.

Phenoxypiperidine derivatives having an antagonistic action on the muscarinic acetylcholine receptor are disclosed, for example, in WO 98/06697; further muscarinic antagonists are disclosed in U.S. Pat. No. 6,037,352. Substances which bind to the nicotinic acetylcholine receptor are described, for example, in WO 00/42044 and EP 0 955 301 A2.

The nicotinic acetylcholine receptors can be divided into two principal main classes, depending on the sites at which they occur.

These are firstly the neuromuscular receptors. These are sub-divided into (α₁α₁βεδ) and (α₁α₁βγδ) receptors. Secondly, there are the neuronal nicotinic acetylcholine receptors, which are found in the ganglia. In these, a distinction is made between the (β₂-β₅) receptors and the (α₂-α₉) receptors, in this respect see also “Basic Neurochemistry”, Ed. Siegel et. al., Raven Press, New York 1993.

The substances of the formula I are capable of interacting with all receptors in this receptor class. The substances of the formula I interact particularly well with the nicotinic α₇ receptor.

In-vitro detection of the interaction with the nicotinic α₇ receptor can be carried out, for example, analogously to J. M. Ward et al, FEBS 1990, 270, 45-48 or D. R. E. Macallan, FEB 1998, 226, 357-363.

Further in-vitro tests for nicotinic receptors are described in F. E. D'Amour et al, Manual for Laboratory Work in Mammalian Physiology, 3rd Ed., The University of Chicago Press (1965), W. Sihver et al, Neuroscience 1998, 85, 1121-1133 or B. Latli et al, J. Med. Chem. 1-999, 42, 2227-22234.

Of the muscarinic acetylcholine receptors, sub-types m1, m2, m3 and m4 are known.

Interactions of substances with the muscarinic receptors m1 and m2 can be determined, for example, by means of the ³H-QNB (quinuclidinyl benzilate) inhibition test. The test is carried out as described by Yamamura and Snyder (Yamamura, H. I. and Snyder S. H., Proc Nat Acad Sci USA 1974; 71: 1725-9): in this test, rat brain is homogenised in 400 vol (w/v) of 0.32 M sucrose and subsequently centrifuged at 1000×g for 10 min at 2° C. 100 μl of the supernatant are incubated with 0.4 nM ³H-QNB in a total volume of 500 μl (50 mM phosphate buffer, pH 7.4) at 25° C. for 1 h. Nonspecific binding is determined with 1 μM QNB.

The compounds of the formula I and physiologically acceptable salts thereof can be used for the prophylaxis or treatment of diseases of the central nervous system in which binding to the nicotinic and/or muscarinic acetylcholine receptor leads to an improvement in the clinical picture.

These diseases include schizophrenia, depression, anxiety states, dementia, in particular Alzheimer's disease and Lewy bodies dementia, neuro-degenerative diseases, Parkinson's disease, Huntington's disease, Tourette's syndrome, learning and memory impairments, age-related memory impairment, and amelioration of withdrawal symptoms in nicotine dependence. Owing to the neuroprotective action, compounds of the formula I are used in strokes and brain damage by toxic compounds.

In the treatment of the diseases described, the compounds according to the invention can also be employed in combination with other pharmacologically effective compounds, such as, for example, with the substances disclosed in WO 98/06697. The compounds according to the invention are given either simultaneously or before or after the other substances mentioned.

Compounds of the formula I and salts and solvates thereof are also suitable as intermediates for the preparation of other medicament active ingredients.

The invention also relates to the stereoisomers (enantiomers and racemates thereof as well as diastereomers), hydrates and solvates of these compounds. The term solvates of the compounds is taken to mean adductions of inert solvent molecules onto the compounds which form owing to their mutual attractive force. Solvates are, for example, mono- or dihydrates or alcoholates.

The term pharmaceutically usable derivatives is taken to mean, for example, the salts of the compounds according to the invention and also so-called prodrug compounds.

The term prodrug derivatives is taken to mean compounds of the formula I modified with, for example, alkyl or acyl groups, sugars or oligopeptides which are rapidly cleaved in the organism to give the effective compounds according to the invention.

These also include biodegradable polymer derivatives of the compounds according to the invention, as described, for example, in Int. J. Pharm. 115, 61-67 (1995).

The invention also relates to mixtures of the compounds of the formula I according to the invention, for example mixtures of two diastereomers, for example in the ratio 1:1, 1:2, 1:3, 1:4, 1:5, 1:10, 1:100 or 1:1000.

Particular preference is given here to mixtures of stereoisomeric compounds.

The invention relates to the compounds of the formula I and physiologically acceptable acid-addition salts thereof. The invention also relates to the solvates, for example hydrates or alcoholates, of these compounds.

The invention also relates to a process for the preparation of compounds of the formula I and pharmaceutically usable derivatives, salts and solvates thereof, characterised in that the following reaction steps are carried out:
a) A compound of the formula V 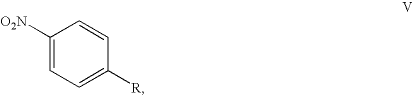
   in which R is a nucleophilic leaving group usually employed in nucleophilic substitutions on aromatic compounds, such as, for example, F, Cl, Br or 1, is brought to reaction with a compound of the formula VI 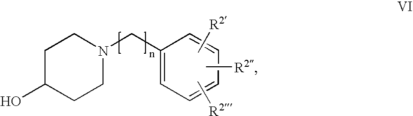
   in which R²′, R²″ R²′″and n are as defined in claim 1, giving a compound of the formula IV 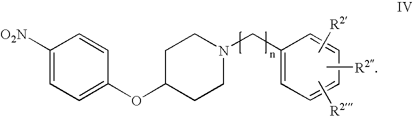
b) The resultant phenoxy-piperidine of the formula IV is subsequently converted by hydrogenation and optionally alkylation into a compound of the formula II 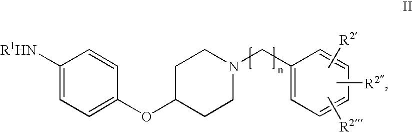
   in which R¹ is as defined in claim 1, which is then
c) reacted further with a compound of the formula III 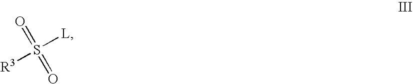
   in which R³ is as defined in claim 1, and L is a nucleophilic leaving group known per se, preferably Hal and particularly preferably Cl, giving a compound of the formula 1.

As a process variant, the sulfonation in accordance with step (c) can also be carried out before the alkylation in accordance with step (b).

A resultant base of the formula I is converted into one of its salts by treatment with an acid.

The invention additionally relates to the hydroxypiperidines of the formula VI and the phenoxypiperidines of the formula IV as intermediate compounds for the preparation of the compounds of the formula 1.

The invention also relates to the compounds of the formula I according to claim 1 and pharmaceutically acceptable derivatives, salts or solvates thereof as medicaments.

The invention likewise relates to the compounds of the formula I according to claim 1 and pharmaceutically acceptable derivatives, salts or solvates thereof as effectors of the nicotinic acetylcholine receptor.

The invention likewise relates to the compounds of the formula I according to claim 1 and pharmaceutically acceptable derivatives, salts or solvates thereof as effectors of the muscarinic acetylcholine receptor.

The invention furthermore relates to the medicament active ingredients according to the invention as nicotinic acetylcholine receptor effectors and/or muscarinic acetylcholine receptor effectors for the prophylaxis or treatment of schizophrenia, depression, anxiety states, dementia, Alzheimer's disease, Lewy bodies dementia, neurodegenerative diseases, Parkinson's disease, Huntington's disease, Tourette's syndrome, learning and memory impairments, age-related memory impairment, amelioration of withdrawal symptoms in nicotine dependence, strokes or brain damage by toxic compounds.

The invention furthermore relates to the use of compounds of the formula I for the preparation of medicaments, in particular medicaments which are employed for the treatment of diseases based on dysfunction of nicotinic and/or muscarinic acetylcholine receptors.

The invention likewise relates to the use of compounds of the formula I according to claim 1 and/or physiologically acceptable salts or solvates thereof for the preparation of a medicament, in particular for the preparation of a medicament for the prophylaxis or treatment of diseases in which in which the binding of one or more active ingredients present in the said medicament to nicotinic and/or muscarinic acetylcholine receptors leads to an improvement in the clinical picture.

The invention furthermore relates to the use of compounds of the formula I according to claim 1 and/or of physiologically acceptable salts and solvates thereof for the preparation of a medicament for the prophylaxis or treatment of schizophrenia, depression, anxiety states, dementia, Alzheimer's disease, Lewy bodies dementia, neurodegenerative diseases, Parkinson's disease, Huntington's disease, Tourette's syndrome, learning and memory impairments, age-related memory impairment, amelioration of withdrawal symptoms in nicotine dependence, strokes or brain damage by toxic compounds.

Finally, the invention relates to pharmaceutical compositions comprising the compounds of the formula I and pharmaceutically acceptable derivatives, salts or solvates thereof, and to a process for the preparation of the pharmaceutical compositions.

The compounds of the formula I may have one or more centres of chirality and therefore occur in various stereoisomeric forms. The formula I includes all these forms.

For all radicals which can occur more than once, such as, for example, A, R² or R⁴, their meanings are independent of one another.

A is alkyl, is unbranched (linear) or branched, and has 1, 2, 3, 4, 5, 6, 7, 8, 9 or 10 carbon atoms.

A is preferably methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl.

A is very particularly preferably alkyl having 1-6 carbon atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl. Furthermore, A is cycloalkyl, preferably cyclopropyl, cyclobutyl, cyclopentyl, cyclohexyl, cycloheptyl, cyclooctyl or 2,6,6-trimethylbicycle[3.1.1]heptyl, but likewise mono- or bicyclic terpenes, preferably p-methane, menthol, pinane, bornane or camphor, where each known stereoisomeric form is incuded, or adamantyl. For camphor, this means both L-camphor and Dcamphor.

Ar is phenyl, naphthyl or biphenyl, each of which is unsubstituted or mono- or polysubstituted by Hal, A, OR⁵, N(R⁵)₂, NO₂, CN, COOR⁵, CON(R⁵)₂, NR⁵COR⁵, NR⁵CON(R⁵)₂, NR⁵SO₂A, COR⁵, SO₂NR⁵, SO₂NR⁵ or SO₂A, where A has one of the meanings indicated above, and R⁵ and m have one of the meanings indicated below.

Ar is preferably unsubstituted or substituted phenyl, naphthyl or biphenyl, specifically preferably phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-trifluoromethylphenyl, o-, m- or p-aminophenyl, o-, m- or p-hydroxyphenyl, o-, m- or p-nitrophenyl, o-, m- or p-(trifluoromethoxy)phenyl, o-, m- or p-cyanophenyl, o-, m- or p-methoxyphenyl, o-, m- or p-ethoxyphenyl, o-, m- or p-fluorophenyl, o-, m- or p-bromophenyl, o-, m- or p-chlorophenyl, o-, m- or p-(difluoromethoxy)phenyl, o-, m- or p-(fluoromethoxy)phenyl, furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2-chloro-3-methyl-, 2-chloro-4-methyl-, 2-chloro-5-methyl-, 2-chloro-6-methyl-, 2-methyl-3-chloro-, 2-methyl-4-chloro-, 2-methyl-5-chloro-, 2-methyl-6-chloro-, 3-chloro-4-methyl-, 3-chloro-5-methyl- or 3-methyl-4-chlorophenyl, 2-bromo-3-methyl-, 2-bromo-4-methyl-, 2-bromo-5-methyl-, 2-bromo-6-methyl-, 2-methyl-3-bromo-, 2-methyl-4-bromo-, 2-methyl-5-bromo-, 2-methyl-6-bromo-, 3-bromo-4-methyl-, 3-bromo-5-methyl- or 3-methyl-4-bromophenyl, 2,4- or 2,5-dinitrophenyl, 2,5- or 3,4-dimethoxyphenyl, 3-nitro-4-chlorophenyl, 2,3,4-, 2,3,5-, 2,3,6-, 2,4,6- or 3,4,5-trichlorophenyl, 2,4,6-tri-tert-butylphenyl, furthermore preferably 2-nitro-4-(trifluoromethyl)phenyl, 3,5-di(trifluoromethyl)phenyl, 2,5-dimethylphenyl, 2-hydroxy-3,5-dichlorophenyl, 2-fluoro-5- or 4-fluoro-3-(trifluoromethyl)phenyl, 4-chloro-2- or 4-chloro-3-(trifluoromethyl)-, 2-chloro-4 or 2-chloro-5-(trifluoromethyl)phenyl, 4-bromo-2- or 4-bromo-3-(trifluoromethyl)phenyl, p-iodophenyl, 2-nitro-4-methoxyphenyl, 2,5-dimethoxy-4-nitrophenyl, 2-methyl-5-nitrohenyl, 2,4-dimethyl-3-nitrophenyl, 4-fluoro-3-chlorophenyl, 4-fluoro-3,5-dimethylphenyl, 2-fluoro-4-bromophenyl, 2,5-difluoro-4-bromophenyl, 2,4-dichloro-5-methylphenyl, 3-bromo-6-methoxyphenyl, 3-chloro-6-methoxyphenyl, 2-methoxy-5-methylphenyl or 2,4,6-triisopropylphenyl, 2-, 3 or 4-methoxycarbonylphenyl, 2-, 3 or 4-ethoxycarbonylphenyl, 2-, 3 or 4-propoxycarbonylphenyl, 2-, 3 or 4-butoxycarbonylphenyl, 2-, 3 or 4-pentoxycarbonylphenyl, 2-, 3 or 4-hexoxycarbonylphenyl, 2-, 3 or 4-methylaminocarbonylphenyl, 2-, 3 or 4-ethylaminocarbonylphenyl, 2-, 3 or 4-propylaminocarbonylphenyl, 2-, 3 or 4-butylaminocarbonylphenyl, 2-, 3 or 4-pentylaminocarbonylphenyl, 2-, 3 or 4-hexylaminocarbonylphenyl, 2,3-, 2,4- or 2,5-dimethylaminocarbonylphenyl or 2,3-, 2,4- or 2,5-diethylaminocarbonylphenyl.

Ar is particularly preferably phenyl or o-methoxyphenyl.

A-Ar is arylalkyl, where A and Ar have one of the meanings indicated above A-Ar is preferably benzyl, phenylethyl, phenylpropyl, phenylbutyl, phenylpentyl, phenylhexyl, phenylheptyl, naphthylmethyl, naphthylethyl, naphthylpropyl or naphthylbutyl. A-Ar is particularly preferably benzyl or phenylethyl.

Hal is fluorine, chlorine, bromine or iodine, particularly preferably fluorine, chlorine or bromine.

R¹ is hydrogen or A, where A has one of the meanings indicated above. R¹ is preferably hydrogen, methyl, ethyl, n-propyl, i-propyl, n-butyl, i-butyl or t-butyl. R¹ is particularly preferably hydrogen.

R²′, R²″ and R²′″ are each, independently of one another, H, A, OH, OCH₃, OCF₃, Hal, CN, COOR¹, CONR¹ or NO₂, where A, Hal and R¹ have one of the above-mentioned meanings. R²′, R²″ and R²′″ are, in particular, hydrogen, hydroxyl, methoxy, ethoxy, propoxy, butoxy, pentyloxy, hexyloxy, trifluoromethoxy, fluorine, chlorine, bromine, iodine, cyano, nitro, methoxycarbonyl, ethoxycarbonyl, propoxycarbonyl, butoxycarbonyl, pentoxycarbonyl, hexoxycarbonyl, methylaminocarbonyl, ethylaminocarbonyl, propylaminocarbonyl, butylaminocarbonyl, pentylaminocarbonyl or hexylaminocarbonyl.

R²′, R²″ and R²′″ are particularly preferably hydrogen.

R³ is A, Ar or A-Ar, where A, Ar and A-Ar have one of the above-mentioned meanings. R³ is in particular methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, trifluoromethyl, pentafluoroethyl or 2,2,2-trifluoroethyl, phenyl, o-, m- or p-tolyl, o-, m- or p-ethylphenyl, o-, m- or p-propylphenyl, o-, m- or p-isopropylphenyl, o-, m- or p-tert-butylphenyl, o-, m- or p-trifluoromethylphenyl, o-, m- or p-aminophenyl, o-, m- or p-hydroxyphenyl, o-, m- or p-nitrophenyl, o-, m- or p-(trifluoromethoxy)phenyl, o-, m- or p-cyanophenyl, o-, m- or p-methoxyphenyl, o-, m- or p-ethoxyphenyl, o-, m- or p-fluoro-phenyl, o-, m- or p-bromophenyl, o-, m- or p-chlorophenyl, o-, m- or p-(difluoromethoxy)phenyl, o-, m- or p-(fluoromethoxy)phenyl, furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2-chloro-3-methyl-, 2-chloro-4-methyl-, 2-chloro-5-methyl-, 2-chloro-6-methyl-, 2-methyl-3-chloro-, 2-methyl-4-chloro-, 2-methyl-5-chloro-, 2-methyl-6-chloro-, 3-chloro-4-methyl-, 3-chloro-5-methyl- or 3-methyl-4-chlorophenyl, 2-bromo-3-methyl-, 2-bromo-4-methyl-, 2-bromo-5-methyl-, 2-bromo-6-methyl-, 2-methyl-3-bromo-, 2-methyl-4-bromo-, 2-methyl-5-bromo-, 2-methyl-6-bromo-, 3-bromo-4-methyl-, 3-bromo-5-methyl- or 3-methyl-4-bromophenyl, 2,4- or 2,5-dinitrophenyl, 2,5- or 3,4-dimethoxyphenyl, 3-nitro-4-chlorophenyl, 2,3,4-, 2,3,5-, 2,3,6-, 2,4,6- or 3,4,5-trichlorophenyl, 2,4,6-tri-tert-butylphenyl, furthermore preferably 2-nitro-4-(trifluoromethyl)phenyl, 3,5-di(trifluoromethyl)phenyl, 2,5-dimethylphenyl, 2-hydroxy-3,5-dichlorophenyl, 2-fluoro-5- or 4-fluoro-3-(trifluoromethyl)phenyl, 4-chloro-2- or 4-chloro-3-(trifluoromethyl)-, 2-chloro-4- or 2-chloro-5-(trifluoromethyl)phenyl, 4-bromo-2- or 4-bromo-3-(trifluoromethyl)phenyl, p-iodophenyl, 2-nitro-4-methoxyphenyl, 2,5-dimethoxy-4-nitrophenyl, 2-methyl-5-nitrophenyl, 2,4-dimethyl-3-nitrophenyl, 4-fluoro-3-chlorophenyl, 4-fluoro-3,5-dimethylphenyl, 2-fluoro-4-bromophenyl, 2,5-difluoro-4-bromophenyl, 2,4-dichloro-5-methylphenyl, 3-bromo-6-methoxyphenyl, 3-chloro-6-methoxyphenyl, 2-methoxy-5-methylphenyl or 2,4,6-triisopropylphenyl, benzyl, 2-, 3- or 4-nitrophenylmethyl, 2,3-, 2,4-, 2,5-, 2,6-, 3,4- or 3,5-dinitrophenylmethyl, 2,3,4-, 2,3,5-, 2,3,6-, 2,4,5-, 2,4,6- or 3,4,5-trinitrophenylmethyl, phenylpropyl, phenylbutyl, phenylpentyl, phenylhexyl, phenylheptyl, naphthylmethyl, naphthylethyl, naphthylpropyl or naphthylbutyl.

R³ is particularly preferably 2,2,2-trifluoroethyl, n-propyl, i-propyl, n-butyl, phenyl, benzyl or 2-nitrophenylmethyl.

n is 0, 1, 2, 3, 4, 5, 6, 7, 8, 9 or 10. n is preferably 0, 1, 2, 3, 4 or 5. n is particularly preferably =1.

The invention relates in particular to the compounds of the formula I in which at least one of the said radicals has one of the preferred meanings indicated above. The following principle applies here for a given compound of the formula I: the more of the radicals present therein have a preferred meaning, the more preferred the compound is overall. Some preferred groups of compounds can be expressed by the following sub-formulae Ia to Ij, which conform to the formula I and in which the radicals not designated in greater detail have the meaning indicated for the formula 1, but in which

| | | |
|---|---|---|
| in la | R¹ | is hydrogen; |
| in lb | R²′, R²″ and R²′″ | are hydrogen; |
| in lc | R³ | is n-propyl, i-propyl, n-butyl, 2,2,2- |
| | | trifluoroethyl, phenyl, benzyl or 2- |
| | | nitrophenylmethyl; |
| in ld | R³ | is i-propyl; |
| in le | R³ | is benzyl; |
| in lf | n | is 1; |
| in lg | n | is 0 or 1 and |
| | R¹ | is hydrogen, methyl or ethyl; |
| In lh | n | is 0 or 1, |
| | R¹ | is hydrogen, methyl or ethyl and |
| | R²′, R²″, R²′″ | are each, independently of one another, |
| | | hydrogen, Hal, methyl or methoxy; |
| In li | n | is 0 or 1, |
| | R¹ | is hydrogen, methyl or ethyl, |
| | R²′, R²″, R²′″ | are each, independently of one another, |
| | | hydrogen, Hal, methyl or methoxy and |
| | R³ | is n-propyl, i-propyl, n-butyl, 2,2,2- |
| | | trifluoroethyl, phenyl, benzyl or 2- |
| | | nitrophenylmethyl; |
| ln lj | n | is 0 or 1, |
| | R¹ | is hydrogen, methyl or ethyl, |
| | R²′, R²″, R²′″ | are each, independently of one another, |
| | | hydrogen, Hal, methyl or methoxy and |
| | R³ | is i-propyl or benzyl. |

The invention relates in particular to the following compounds of the formula I:
a) N-[4-(1-benzylpiperidin-4-yloxy)phenyl]-C-phenylmethanesulfonamide,
b) N-[4-(1-benzylpiperidin-4-yloxy)phenyl]-C-[2-nitrophenyl]methanesulfonamide,
c) N-[4-(1-benzylpiperidin-4-yloxy)phenyl]benzenesulfonamide,
d) N-[4-(1-benzylpiperidin-4-yloxy)phenyl]-2-propanesulfonamide,
e) N-[4-(1-benzylpiperidin-4-yloxy)phenyl]-1-butanesulfonamide,
f) N-[4-(1-benzylpiperidin-4-yloxy)phenyl]-1-propanesulfonamide,
g) N-[4-(1-benzylpiperidin-4-yloxy)phenyl]-1-2,2,2-trifluoroethanesulfonamide
   and pharmaceutically usable derivatives, solvates and stereoisomers thereof, including mixtures thereof in all ratios.

The compounds of the formula I and also the starting materials for their preparation are prepared by methods known per se, as described in the literature (for example in standard works, such as Houben-Weyl, Methoden der Organischen Chemie [Methods of Organic Chemistry], Georg Thieme Verlag, Stuttgart; Organic Reactions, John Wiley & Sons, Inc., New York), to be precise under reaction conditions as are known and suitable for the said reactions. Use can also be made here of variants which are known per se, but are not mentioned here in greater detail.

The starting materials for the claimed process can also be formed in situ by not isolating them from the reaction mixture, but instead immediately converting them further into the compounds of the formula 1. On the other hand, it is possible to carry out the reaction stepwise.

The phenoxy-piperidines of the formula I can preferably be obtained by reacting nitrohalobenzenes of the formula V with piperidines of the formula VI to give phenoxy-piperidines of the formula IV, which, after hydrogenation and optionally alkylation, are reacted with suitable sulfonyl compounds of the formula II, such as, for example, the corresponding sulfonyl chlorides.

The nitrobenzene derivatives of the formula V are generally known and commercially available; the compounds of the formula V which are not known can easily be prepared analogously to the known compounds. The corresponding situation applies to the phenoxy-piperidines of the formula VI: these compounds are known or can preferably be prepared by reaction of hydroxypiperidines with benzyl halides.

The reaction of compounds of the formula V with compounds of the formula VI is preferably carried out as follows: a hydroxypiperidine of the formula VI is dissolved in DMF, and 1.5 equivalents of a strong base, such as sodium hydride, sodium ethoxide or potassium tert-butoxide (preferably potassium tert-butoxide) are added. The mixture is stirred at room temperature for approximately one hour, and a nitro compound of the formula V dissolved in DMF is then added dropwise. The mixture is stirred at room temperature for a further hour, and water is then added. The crystals are filtered off with suction, washed and optionally recrystallised.

The hydrogenation of the nitro compounds of the formula IV to give the corresponding amine is usually carried out in accordance with standard procedures of organic chemistry using a suitable hydrogenation catalyst, preferably Ra Ni, in a polar, protic solvent, such as, for example, methanol, at standard or increased pressure and temperatures of from 20 to 200° C., preferably at room temperature.

After the reaction, the solvent is removed, and the residue is reacted further.

For the preparation of the variants of the compounds of the formula I in which the radical R² is not hydrogen, this is followed by an alkylation, which can be carried out, for example, in accordance with the Leuckart-Wallach reaction, a standard method for the alkylation of amines.

The compounds of the formula II obtained after hydrogenation and optionally alkylation are finally reacted with the sulfone compounds of the formula III to give the phenoxy-piperidine-sulfonamides of the formula 1.

The alkylation can equally well be delayed until after the sulfonation with deprotonation of the sulfonamide using suitable alkylating agents, such as, for example, alkyl iodide.

The reactions described above are generally carried out in an inert solvent, in the presence of an acid-binding agent, preferably an organic base, such as triethylamine, dimethylaniline, pyridine or quinoline, an alkali or alkaline-earth metal hydroxide, carbonate or bicarbonate or another salt of a weak acid of the alkali or alkaline-earth metals, preferably of potassium, sodium, calcium or caesium.

Examples of suitable inert solvents for the above-described reactions are hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, N-methylpyrrolidone (NMP), dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Depending on the conditions used, the reaction temperature for the above-described reactions is between about −10° and 150°, normally between 0° and 130°, preferably between 0° and 50°, particularly preferably room temperature.

Depending on the conditions used, the reaction time is between a few minutes and several days.

A base of the formula I obtained can be converted into the associated acid-addition salt using an acid. Suitable acids for this reaction are those which give physiologically acceptable salts. Thus, it is possible to use inorganic acids, for example sulfuric acid, hydrohalic acids, such as hydrochloric acid or hydrobromic acid, phosphoric acids, such as orthophosphoric acid, nitric acid, sulfamic acid, furthermore organic acids, specifically aliphatic, alicyclic, araliphatic, aromatic or heterocyclic monobasic or polybasic carboxylic, sulfonic or sulfuric acids, such as formic acid, acetic acid, propionic acid, pivalic acid, diethylacetic acid, malonic acid, succinic acid, pimelic acid, fumaric acid, maleic acid, lactic acid, tartaric acid, malic acid, benzoic acid, salicylic acid, 2-phenylpropionic acid, citric acid, gluconic acid, ascorbic acid, nicotinic acid, isonicotinic acid, methane- or ethanesulfonic acid, ethanedisulfonic acid, 2-hydroxyethanesulfonic acid; benzenesulfonic acid, p-toluenesulfonic acid, naphthalenemorio- and -disulfonic acids and laurylsulfuric acid.

The free bases of the formula I can, if desired, be liberated from their salts by treatment with strong bases, such as sodium hydroxide, potassium hydroxide, sodium carbonate or potassium carbonate, so long as no further acidic groups are present in the molecule.

Compounds of the formula I can furthermore be obtained by liberating compounds of the formula I from one of their functional derivatives by treatment with a solvolysing or hydrogenolysing agent.

Preferred starting materials for the solvolysis or hydrogenolysis are those which conform to the formula 1, but contain corresponding protected amino and/or hydroxyl groups instead of one or more free amino and/or hydroxyl groups, preferably those which carry an amino-protecting group instead of an H atom bonded to an N atom, in particular those which carry an R′-N group, in which R′ is an amino-protecting group, instead of an HN group, and/or those which carry a hydroxyl-protecting group instead of the H atom of a hydroxyl group, for example those which conform to the formula 1, but carry a —COOR″ group, in which R″ is a hydroxyl-protecting group, instead of a —COOH group.

Preferred starting materials are also the oxadiazole derivatives, which can be converted into the corresponding amidino compounds.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size are furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alka noyl, such as acetyl, propionyl and butyryl; aralkanoyl, such as phenylacetyl; aroyl, such as benzoyl and tolyl; aryloxyalkanoyl, such as POA; alkoxycarbonyl, such as methoxycarbonyl, ethoxycarbonyl, 2,2,2-trichloroethoxycarbonyl, BOC (tert-butoxycarbonyl) and 2-iodoethoxycarbonyl; aralkoxycarbonyl, such as CBZ (“carbobenzoxy”), 4-methoxybenzyloxycarbonyl and FMOC; and arylsulfonyl, such as Mtr. Preferred amino-protecting groups are BOC and Mtr, furthermore CBZ, Fmoc, benzyl and acetyl.

The term “hydroxyl-protecting group” is likewise known in general terms and relates to groups which are suitable for protecting a hydroxyl group against chemical reactions, but which are easy to remove after the desired chemical reaction has been carried out elsewhere in the molecule. Typical of such groups are the above-mentioned unsubstituted or substituted aryl, aralkyl or acyl groups, furthermore also alkyl groups. The nature and size of the hydroxyl-protecting groups are not crucial since they are removed again after the desired chemical reaction or reaction sequence; preference is given to groups having 1-20, in particular 1-10, carbon atoms. Examples of hydroxyl-protecting groups are, inter alia, benzyl, 4-methoxybenzyl, p-nitrobenzoyl, p-toluenesulfonyl, tert-butyl and acetyl, where benzyl and tert-butyl are particularly preferred.

The compounds of the formula I are liberated from their functional derivatives—depending on the protecting group used—for example using strong acids, advantageously using TFA or perchloric acid, but also using other strong inorganic acids, such as hydrochloric acid or sulfuric acid, strong organic carboxylic acids, such as trichloroacetic acid, or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 50°, preferably between 15 and 300 (room temperature).

The BOC, OBut and Mtr groups can, for example, preferably be cleaved off using TFA in dichloromethane or using approximately 3 to 5N HCl in dioxane at 15-30°, the FMOC group using an approximately 5 to 50% solution of dimethylamine, diethylamine or piperidine in DMF at 15-30°.

Hydrogenolytically removable protecting groups (for example CBZ, benzyl or the liberation of the amidino group from its oxadiazole derivative)) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 100° and pressures between about 1 and 200 bar, preferably at 20-30° and 1-10 bar. Hydrogenolysis of the CBZ group succeeds well, for example, on 5 to 10% Pd/C in methanol or using ammonium formate (instead of hydrogen) on Pd/C in methanol/DMF at 20-30°.

Esters can be saponified, for example, using acetic acid or using NaOH or KOH in water, water/THF or water/dioxane, at temperatures between 0 and 100°.

Free amino groups can furthermore be acylated in a conventional manner using an acid chloride or anhydride or alkylated using an unsubstituted or substituted alkyl halide, or reacted with CH₃—C(═NH)—OEt, advantageously in an inert solvent, such as dichloromethane or THF and/or in the presence of a base, such as triethylamine or pyridine, at temperatures between −60 and +30°.

Compounds of the formula I according to the invention may be chiral owing to their molecular structure and may accordingly occur in various enantiomeric forms. They can therefore exist in racemic or in optically active form. Since the pharmaceutical activity of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitably N-protected amino acids (for example N-benzoylproline or N-benzenesulfonylproline), or the various optically active camphorsulfonic acids. Also advantageous is chromatographic enantiomer resolution with the aid of an optically active resolving agent (for example dinitrobenzoylphenylglycine, cellulose triacetate or other derivatives of carbohydrates or chirally derivatised methacrylate polymers immobilised on silica gel). Suitable eluents for this purpose are aqueous or alcoholic solvent mixtures, such as, for example, hexane/isopropanol/acetonitrile, for example in the ratio 82:15:3.

The invention furthermore relates to the use of the compounds of the formula I and/or physiologically acceptable salts thereof for the preparation of a medicament (pharmaceutical composition), in particular by non-chemical methods. They can be converted here into a suitable dosage form together with at least one solid, liquid and/or semi-liquid excipient or adjuvant and, if desired, in combination with one or more further active ingredients.

These compositions can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatine, carbohydrates, such as lactose or starch, magnesium stearate, talc or Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, to prepare injection compositions. The compositions indicated may be sterilised and/or comprise adjuvants, such as lubricants, preservatives, stabilisers and/or wetting agents, emulsifying agents, salts for modifying the osmotic pressure, buffer substances, colorants and flavours and/or a plurality of further active ingredients, for example one or more vitamins.

In general, the substances according to the invention are administered analogously to known, commercially available compositions, preferably in doses between about 5 mg and 100 mg, in particular between 10 and 40 mg per dosage unit. The daily dose is preferably between about 0.5 and 1 mg/kg of body weight.

The specific dose for each individual patient depends on a wide variety of factors, for example on the efficacy of the specific compound employed, on the age, body weight, general state of health, sex, on the diet, on the time and method of administration, on the excretion rate, medicament combination and severity of the particular disease to which the therapy applies. Oral administration is preferred.

The invention thus also relates to medicaments comprising at least one compound of the formula I and/or pharmaceutically usable derivatives, solvates and stereoisomers thereof, including mixtures thereof in all ratios.

The invention furthermore relates to medicaments comprising at least one compound of the formula I and/or pharmaceutically usable derivatives, solvates and stereoisomers thereof, including mixtures thereof in all ratios, and at least one further medicament active ingredient.

The invention also relates to a set (kit) consisting of separate packs of
(a) an effective amount of a compound of the formula I and/or pharmaceutically usable derivatives, solvates and stereoisomers thereof, including mixtures thereof in all ratios, and
(b) an effective amount of a further medicament active ingredient.

The set comprises suitable containers, such as boxes, individual bottles, bags or ampoules. The set may, for example, comprise separate ampoules each containing an effective amount of a compound of the formula I and/or pharmaceutically usable derivatives, solvates and stereoisomers thereof, including mixtures thereof in all ratios,

and an effective amount of a further medicament active ingredient in dissolved or lyophilised form.

The invention furthermore relates to the use of compounds of the formula I and/or pharmaceutically usable derivatives, solvates and stereoisomers thereof, including mixtures thereof in all ratios,

for the preparation of a medicament for the prophylaxis or treatment of schizophrenia, depression, anxiety states, dementia, Alzheimer's disease, Lewy bodies dementia, neurodegenerative diseases, Parkinson's disease, Huntington's disease, Tourette's syndrome, learning and memory impairments, age-related memory impairment, amelioration of withdrawal symptoms in nicotine dependence, strokes or brain damage by toxic compounds,

in combination with at least one further medicament active ingredient.

Even without further comments, it is assumed that a person skilled in the art will be able to utilise the above description in the broadest scope. The preferred embodiments should therefore merely be regarded as descriptive disclosure which is absolutely not limiting in any way.

Above and below, all temperatures are given in ° C. In the following examples, “conventional work-up” means that, if necessary, the solvent is removed, water is added if necessary, the pH is, if necessary, adjusted to between 2 and 10, depending on the constitution of the end product, the mixture is extracted with ethyl acetate or dichloromethane, the phases are separated, the organic phase is washed with NaCl solution, dried over sodium sulfate, filtered and evaporated, and the product is purified by chromatography by means of preparative HPLC:

| | |
|---|---|
| Column: | RP 18 (15 μm) Lichrosorb 250 × 50 |
| Mobile phase: | A: 98H20; 2CH3CN; 0.1% TFA |
| | B: 10H20; 90CH3CN; 0.1% TFA |
| UV detection: | 250 nm |
| Flow rate: | 10 ml/min |
| Mass spectrometry (MS): | ESI (electrospray ionisation) (M + H)⁺ |
| | EI (electron impact ionisation) (M⁺) |

### EXAMPLE 1

### Synthesis of the Precursor

1 g of 1-benzyl-4-(4-nitrophenoxy)piperidine is dissolved in 30 ml of methanol and hydrogenated by standard procedures using 1 g of Ra Ni/H₂. The mixture is filtered off and dried in a rotary evaporator: 4-(1-benzylpiperidin-4-yloxy)phenylamine.
DC in methanol, Rf=0.73; EI-MS (M⁺) 282.

### EXAMPLE 2

200 mg of 4-(1-benzylpiperidin-4-yloxy)phenylamine and 162 mg of phenylmethylsulfonyl chloride are dissolved in 10 ml of DMF, and 0.25 ml of triethylamine is added. The mixture is stirred at room temperature for 14 h. Conventional work-up is carried out: N-[4-(1-benzylpiperidin-4-yloxy)phenyl]-C-phenylmethanesulfonamide.
DC in methanol, Rf=0.40; HPLC-ESI-MS (M+H)⁺437.

### EXAMPLE 3

Analogously to Example 2, reaction of 4-(1-benzylpiperidin-4-yloxy)phenylamine with
a) phenylsulfonyl chloride gives:
N-[4-(1-benzylpiperidin-4-yloxy)phenyl]benzenesulfonamide.
DC in methanol, Rf=0.58; EI-MS (M)⁺422.
b) (2-nitrophenyl)methanesulfonyl chloride gives:
N-[4-(1-benzylpiperidin-4-yloxy)phenyl]-C-[2-nitrophenyl]methane sulfonamide.
DC in methanol, Rf=0.32; HPLC-ESI-MS (M+H)⁺482.
c) propane-2-sulfonyl chloride gives:
N-[4-(1-benzylpiperidin-4-yloxy)phenyl]propane-2-sulfonamide.
DC in methanol, Rf=0.63; HPLC-ESI-MS (M+H)⁺425.
d) butane-1-sulfonyl chloride gives:
N-[4-(1-benzylpiperidin-4-yloxy)phenyl]butane-1-sulfonamide.
e) propane-1-sulfonyl chloride gives:
N-[4-(1-benzylpiperidin-4-yloxy)phenyl]propane-1-sulfonamide.
DC in methanol, Rf=0.63; HPLC-ESI-MS (M+H)⁺425.
f) 2,2,2-trifluoroethane-1-sulfonyl chloride gives:
N-[4-(1-benzylpiperidin-4-yloxy)phenyl]2,2,2-trifluoroethane-1-sulfonamide.
DC in methanol, Rf=0.66; HPLC-ESI-MS (M+H)⁺429.

The following examples relate to pharmaceutical compositions:

### EXAMPLE A

### Injection Vials

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised and sealed under sterile conditions. Each injection vial contains 5 mg of active ingredient.

### EXAMPLE B

### Suppositories

A mixture of 20 g of an active ingredient of the formula I is melted with 100 g of soya lecithin and 1400 g of cocoa butter, poured into moulds and allowed to cool. Each suppository contains 20 mg of active ingredient.

### EXAMPLE C

### Solution

A solution is prepared from 1 g of an active ingredient of the formula I 9.38 g of NaH₂PO₄×2H₂O, 28.48 g of NaH₂PO₄×12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula I are mixed with 99.5 g of Vaseline under aseptic conditions.

### EXAMPLE E

### Tablets

A mixture of 1 kg of active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE F

### Coated tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula I are introduced into hard gelatine capsules in a conventional manner in such a way that each capsule contains 20 mg of the active ingredient.

### EXAMPLE H

### Ampoules

A solution of 1 kg of active ingredient of the formula I in 60 l of bidistilled water is transferred into ampoules, lyophilised under sterile conditions and sealed under aseptic conditions. Each ampoule contains 10 mg of active ingredient.

1. Compounds of the general formula I 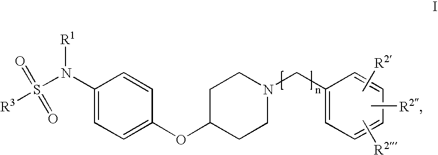
in which
R¹ is H or A,
R²′, R²″, R²′″ are each, independently of one another, H, A, OH, OCH₃, OCF₃, Hal, CN, COOR¹, CONR¹ or NO₂,
R³ is A, Ar or A-Ar,
R⁴ is H or A,
A is unbranched or branched alkyl having 1-10 carbon atoms, in which one or two CH₂ groups may be replaced by O or S atoms and/or by —CH═CH— groups and/or 1-7H atoms may also be replaced by F,
Ar is phenyl, naphthyl or biphenyl, each of which is unsubstituted or mono-, di- or trisubstituted by Hal, A, OR⁴, N(R⁴)₂, NO₂, CN, COOR⁴, CON(R⁴)₂, NR⁴COA, NR⁴CON(R⁴)₂, NR⁴SO₂A, COR⁴, SO₂N(R⁴)₂ or SO₂A,
A-Ar is arylalkyl, where A and Ar have one of the above-mentioned meanings,
Hal is F, Cl, Br or I, and
n is 0, 1, 2, 3, 4, 5, 6, 7, 8, 9 or 10,
and solvates, stereoisomers and pharmaceutically usable derivatives, thereof, including mixtures thereof in all ratios.

2. Compounds according to claim 1, in which
R¹ is hydrogen,
and solvates, stereoisomers and pharmaceutically usable derivatives thereof, including mixtures thereof in all ratios.

3. Compounds according to claim 1, in which
R², R²″, R²′″are hydrogen,
and solvates, stereoisomers and pharmaceutically usable derivatives thereof, including mixtures thereof in all ratios.

4. Compounds according to claim 1, in which R³ is n-propyl, i-propyl, n-butyl, 2,2,2-trifluoroethyl, phenyl, benzyl or 2-nitrophenylmethyl, and solvates, stereoisomers and pharmaceutically usable derivatives thereof, including mixtures thereof in all ratios.

5. Compounds according to claim 1, in which
n is 1,
and solvates, stereoisomers and pharmaceutically usable derivatives thereof, including mixtures thereof in all ratios.

6. Compounds according to claim 1 selected from the group consisting of
N-[4-(1-benzylpiperidin-4-yloxy)phenyl]-C-phenylmethanesulfonamide,
N-[4-(1-benzylpiperidin-4-yloxy)phenyl]-C-[2-nitrophenyl]methanesulfonamide,
N-[4-(1-benzylpiperidin-4-yloxy)phenyl]benzenesulfonamide,
N-[4-(1-benzylpiperidin-4-yloxy)phenyl]-2-propanesulfonamide,
N-[4-(1-benzylpiperidin-4-yloxy)phenyl]-1-butanesulfonamide,
N-[4-(1-benzylpiperidin-4-yloxy)phenyl]-1-propanesulfonamide,
N-[4-(1-benzylpiperidin-4-yloxy)phenyl]-1-2,2,2-trifluoroethanesulfonamide,
and solvates, stereoisomers and pharmaceutically usable derivatives thereof, including mixtures thereof in all ratios.

7. Process for the preparation of compounds of the formula I according to claim 1 and pharmaceutically usable derivatives, solvates and stereoisomers thereof, characterised in that
a) a compound of the formula V 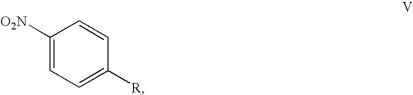
in which R is a nucleophilic leaving group usually employed for nucleophilic substitutions on aromatic compounds, is reacted with a compound of the formula VI 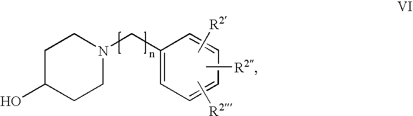
in which R²′, R²″, R²′″ and n are as defined in claim 1, giving a compound of the formula IV 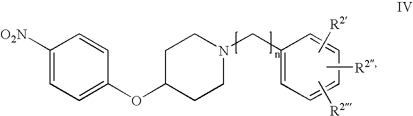
b) the resultant phenoxy-piperidine of the formula IV is converted by hydrogenation and optionally alkylation into a compound of the formula II 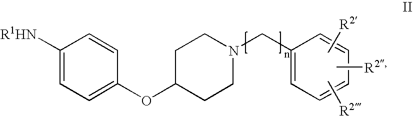
in which R¹ is as defined in claim 1, which is then
c) reacted further with a compound of the formula III
in which R3 is as defined in claim 1, and L is a nucleophilic leaving group known per se, giving a compound of the formula I,
and optionally a protecting group is subsequently cleaved off,
and/or a base or acid of the formula I is converted into one of its salts.

8. Compounds of the formula I and pharmaceutically usable derivatives, solvates and stereoisomers thereof according to claim 1 as effectors of the nicotinic acetylcholine receptor.

9. Compounds of the formula I and pharmaceutically usable derivatives, solvates and stereoisomers thereof according to claim 1 as effectors of the muscarinic acetylcholine receptor.

10. Compounds of the formula I and/or pharmaceutically usable derivatives, solvates and stereoisomers thereof, including mixtures thereof in all ratios, according to claim 1 as medicaments.

11. Medicaments comprising at least one compound of the formula I and/or pharmaceutically usable derivatives, solvates and stereoisomers thereof, including mixtures thereof in all ratios, according to claim 1, and optionally excipients and/or adjuvants.

12. Medicaments comprising at least one compound of the formula I and/or pharmaceutically usable derivatives, solvates and stereoisomers thereof, including mixtures thereof in all ratios, according to, and at least one further medicament active ingredient.

13. Use of compounds according to claim 1 and/or pharmaceutically usable derivatives, solvates and stereoisomers thereof, including mixtures thereof in all ratios, for the preparation of a medicament for the prophylaxis or treatment of diseases in which the binding of one or more active ingredients present in the said medicament to nicotinic and/or muscarinic acetylcholine receptors leads to an improvement in the clinical picture.

14. Use of compounds according to claim 1 and/or pharmaceutically usable derivatives, solvates and stereoisomers thereof, including mixtures thereof in all ratios, for the preparation of a medicament for the prophylaxis or treatment of schizophrenia, depression, anxiety states, dementia, Alzheimer's disease, Lewy bodies dementia, neurodegenerative diseases, Parkinson's disease, Huntington's disease, Tourette's syndrome, learning and memory impairments, age-related memory impairment, amelioration of withdrawal symptoms in nicotine dependence, strokes or brain damage by toxic compounds.

15. Pharmaceutical composition, characterised by a content of at least one compound of the formula I and/or pharmaceutically usable derivatives, solvates and stereoisomers thereof, including mixtures thereof in all ratios, according to claim 1.

16. Process for the preparation of a pharmaceutical compositions comprising at least one compound according to claim 1 comprising converting said composition into a suitable dosage form together with at least one solid, liquid or semi-liquid excipient or adjuvant.

17. Set (kit) consisting of separate packs of
(a) an effective amount of a compound of the formula I according to and/or pharmaceutically usable derivatives, solvates and stereoisomers thereof, including mixtures thereof in all ratios, and
(b) an effective amount of a further medicament active ingredient.

18. Use of compounds of the formula I and/or pharmaceutically usable derivatives, solvates and stereoisomers thereof, including mixtures thereof in all ratios, according to claim 1, for the preparation of a medicament for the prophylaxis or treatment of schizophrenia, depression, anxiety states, dementia, Alzheimer's disease, Lewy bodies dementia, neurodegenerative diseases, Parkinson's disease, Huntington's disease, Tourette's syndrome, learning and memory impairments, age-related memory impairment, amelioration of withdrawal symptoms in nicotine dependence, strokes or brain damage by toxic compounds, in combination with at least one further medicament active ingredient.

19. Intermediate compounds of the formula IV 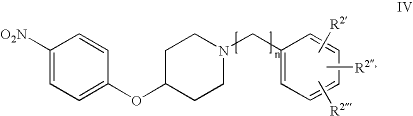
in which R²′, R²″, R²″ and n are as defined in claim 1,
and salts thereof.

20. Intermediate compounds of the formula VI 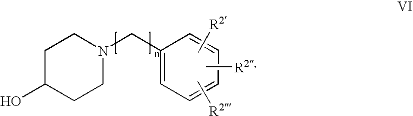
in which R²′, R², R² and n are as defined in claim 1,
and salts thereof.
